(19) United States
(12) Patent Application Publication
(10) Pub. No.: US 2023/0332099 A1
(43) Pub. Date: Oct. 19, 2023

(54) MEDICAL CELL CMU-pb-7 AND APPLICATION THEREOF IN PREPARATION OF ANTIOXIDANT DRUGS

(71) Applicants: Guangdong Xinghai Biotechnology Co., Ltd., Dongguan (CN); Guangdong Xinghai Institute of Cell, Dongguan (CN)

(72) Inventors: Jincheng Zeng, Dongguan (CN); Shaobing Zhang, Dongguan (CN)

(21) Appl. No.: 18/336,974

(22) Filed: Jun. 17, 2023

(30) Foreign Application Priority Data
Aug. 5, 2022 (CN) 202210938301.3

(51) Int. Cl. C12N 1/20; A61K 35/747; A61P 39/06

(52) U.S. Cl. CPC C12N 1/205; A61K 35/747; A61P 39/06; C12R 2001/225

(57) ABSTRACT

The present invention discloses a medical cell CMU-pb-7 and an application thereof in preparation of antioxidant drugs, which belongs to the technical field of biology. The medical cell CMU-pb-7 disclosed by the present invention is a newly isolated and screened strain of Lactobacillus rhamnosus, having a collection number of CCTCC NO: M 2022220. The medical cell CMU-pb-7 of the present invention can enhance the antioxidant capacity of liver tissue, reduce the oxidative stress level, relieve the pathological changes of liver fat, regulate the oxidative stress level in intestinal tract, and relieve the oxidative damage to the ileum of high-fat mice. The medical cell CMU-pb-7 disclosed by the present invention has a great potential application prospect in preparation of antioxidant drugs.

### CROSS REFERENCE TO RELATED APPLICATION

This patent application claims the benefit and priority of Chinese Patent Application No. 2022109383013 filed on Aug. 5, 2022, the disclosure of which is incorporated by reference herein in its entirety as part of the present application.

### TECHNICAL FIELD

The present invention relates to the technical field of biology, and more particularly to a medical cell CMU-pb-7 and an application thereof in preparation of antioxidant drugs.

### BACKGROUND

Human diet structure is directly related to health. A reasonable and healthy diet can form a stable intestinal microecology, enhance the immune function of the body, and enhance the scavenging of metabolites and oxygen free radicals in the body. A long-term high-fat diet will lead to intestinal microecological disorders, affect the normal metabolism of the body, cause excessive reactive oxygen species produced by cell metabolism, induce oxidative stress reaction, weaken the antioxidant function of the body, and further cause a variety of metabolism-related diseases.

Medical cells mainly include functional probiotics, immunocytes, mesenchymal stem cells, etc. Probiotics is a hot topic in current medical cell research. Probiotics are a kind of active microorganisms beneficial to human health. When ingested in sufficient quantities, probiotics can colonize in the intestinal tract of the host, regulate intestinal microecological balance, promote physiological metabolism, and play a probiotic role. At present, medical cell type probiotics mainly include Lactobacillus, Bifidobacterium, saccharomyces, etc. Lactobacillus probiotics have been used more and more widely in clinical applications, and Lactobacillus rhamnosus is one of the common Lactobacillus, which has the functions of regulating intestinal flora and improving body immunity.

Therefore, the problem to be urgently solved by those skilled in the art is to provide a medical cell CMU-pb-7 and an application thereof in preparation of antioxidant drugs.

### SUMMARY

In view of this, the present invention provides a medical cell CMU-pb-7 and an application thereof in preparation of antioxidant drugs.

To achieve the above purpose, the present invention adopts the following technical solution:

A medical cell CMU-pb-7, namely Lactobacillus rhamnosus CMU-pb-7, having a collection number of CCTCC NO: M 2022220, has been preserved in China Center for Type Culture Collection (CCTCC), Wuhan University, Wuhan, China, on Mar. 9, 2022, and is named Lactobacillus rhamnosus CMU-pb-7 taxonomically.

Further, an application of the medical cell CMU-pb-7 in preparation of antioxidant drugs.

The medical cell CMU-pb-7 of the present invention can enhance the antioxidant capacity of liver tissue, reduce the oxidative stress level, relieve the pathological changes of liver fat, regulate the oxidative stress level in intestinal tract, and relieve the oxidative damage to the ileum of high-fat mice.

It can be known from the above technical solution that compared with the prior art, the present invention discloses and provides a medical cell CMU-pb-7 and an application thereof in preparation of antioxidant drugs, wherein the medical cell CMU-pb-7 is a strain of Lactobacillus rhamnosus isolated from feces of healthy people in the laboratory, and CMU-pb-7 can be prepared into antioxidant drugs.

### DESCRIPTION OF DRAWINGS

To more clearly describe the technical solution in the embodiments of the present invention or in the prior art, the drawings required to be used in the description of the embodiments or the prior art will be simply presented below. Apparently, the drawings in the following description are merely the embodiments of the present invention, and for those ordinary skilled in the art, other drawings can also be obtained according to the provided drawings without contributing creative labor.

FIG. 1 is colony morphology of a medical cell CMU-pb-7 of the present invention on an MRS agar plate;

FIG. 2 is colony morphology of a medical cell CMU-pb-7 of the present invention on an anaerobic blood agar plate;

FIG. 3 is morphology of a medical cell CMU-pb-7 of the present invention under a Gram staining microscope (1000×);

FIG. 4 shows GSH level detection results of a high-fat diet mouse model with a medical cell CMU-pb-7 of the present invention;

FIG. 5 shows CAT level detection results of a high-fat diet mouse model with a medical cell CMU-pb-7 of the present invention;

FIG. 6 shows T-AOC level detection results of a high-fat diet mouse model with a medical cell CMU-pb-7 of the present invention;

FIG. 7 shows MDA level detection results of a high-fat diet mouse model with a medical cell CMU-pb-7 of the present invention;

FIG. 8 shows liver tissue morphological observation results of a high-fat diet mouse model with a medical cell CMU-pb-7 of the present invention;

FIG. 9 shows ileum tissue morphological observation results of a high-fat diet mouse model with a medical cell CMU-pb-7 of the present invention;

FIG. 10 shows ileum tissue pathological section HE staining results of a high-fat diet mouse model with a medical cell CMU-pb-7 of the present invention;

FIG. 11 shows liver index influencing results of a high-fat diet mouse with a medical cell CMU-pb-7 of the present invention;

FIG. 12 shows ileum histological injury scores of a high-fat diet mouse with a medical cell CMU-pb-7 of the present invention.

Where, *, P<0.05; **, P<0.01; ***, P<0.001; ****, P<0.0001.

### DETAILED DESCRIPTION

The technical solutions in the embodiments of the present invention will be clearly and fully described below in combination with the drawings in the embodiments of the present invention. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

### Embodiment 1 Isolation, Cultivation and Identification of CMU-pb-7

(1) Sample Source

Healthy people are selected as volunteers. The people selected are required to eat a normal diet for two weeks prior to sampling and have no history of intestinal diseases or antibiotic uses. Feces in the morning of the day are taken as samples. After fresh samples are collected, an intelligent microorganism isolation system of Nanjing FMT medical Co., Ltd. is used for fecal bacteria isolation immediately. After isolation, a crude fecal bacteria solution is collected, added into an MRS broth medium containing 40% of glycerol as a frozen protective solution, and frozen in a −80° C. ultra-low temperature refrigerator for later use.

(2) Isolation of CMU-pb-7

The crude fecal bacteria solution is taken out from the −80° C. refrigerator, and 1 mL of crude fecal bacteria solution is added into 9 mL of normal saline, mixed thoroughly, and gradiently diluted to a concentration of 10⁻³ to 10⁻⁷. 100 μL of bacterial solution of each concentration is drawn, respectively applied to an MRS agar medium, a TPY agar medium and an M17 agar medium, and cultured at 37° C. in anaerobic conditions for 48 h to 72 h. According to the characteristics of colonies, single colonies with a round shape, a smooth surface, a neat edge, a creamy white color and a creamy flavor are selected for pure culture on corresponding agar media.

(3) Cultural Characteristics of CMU-pb-7

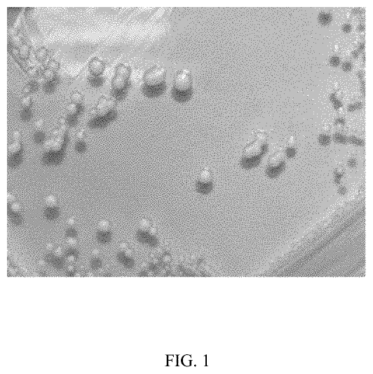

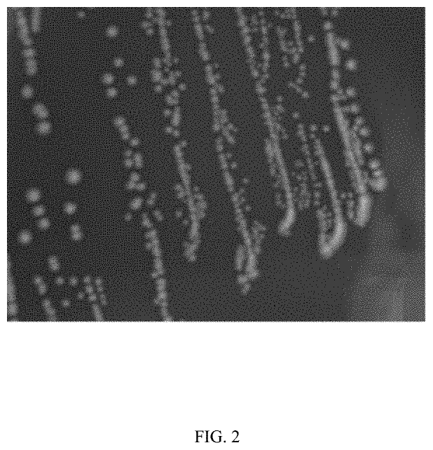

The single colonies of a CMU-pb-7 strain are respectively inoculated on an MRS agar plate and an anaerobic blood agar plate, and cultured at 37° C. in anaerobic conditions for 48 h; the characteristics of the colonies on the MRS plate and the anaerobic blood agar plate are observed, and the results are shown in FIG. 1 to FIG. 2, which show that CMU-pb-7 is a facultative anaerobe, the colonies on the MRS plate and the anaerobic blood agar plate have a round shape, a smooth surface, a neat edge, a creamy white color and a creamy flavor, and no hemolysis is observed on the anaerobic blood agar plate.

(4) Biochemical Identification of CMU-pb-7

Biochemical identification of CMU-pb-7 is carried out by an API 50CHL bacterial biochemical identification system of BioMérieux. Operations are carried out strictly according to the instructions of API 50CHL identification reagent strips, and the reaction results of the strain are obtained and analyzed by API identification software. The identification results show that the strain is Lactobacillus rhamnosus, with an identification rate of 99.9% and a T value of 0.3. The biochemical reaction results of CMU-pb-7 are shown in Table 1.

TABLE 1
Biochemical reaction results of CMU-pb-7
Table 1-Carbohydrate utilization of the
Lactobacillus rhamnosus strain by using API 50CHL

| | | 24 h | 48 h |
|---|---|---|---|
| No. | Substrate | Reaction | Reaction |
| 0 | 0 | − | − |
| 1 | GLY | − | − |
| 2 | ERY | − | − |
| 3 | DARA | − | − |
| 4 | LARA | − | − |
| 5 | RIB | + | + |
| 6 | DXYL | − | − |
| 7 | LXYL | − | − |
| 8 | ADO | − | − |
| 9 | MDX | − | − |
| 10 | GAL | + | + |
| 11 | GLU | + | + |
| 12 | FRU | + | + |
| 13 | MNE | + | + |
| 14 | SBE | + | + |
| 15 | RHA | − | + |
| 16 | DUL | − | + |
| 17 | INO | − | − |
| 18 | MAN | + | + |
| 19 | SOR | + | + |
| 20 | MDM | − | − |
| 21 | MDG | + | + |
| 22 | NAG | + | + |
| 23 | AMY | + | + |
| 24 | ARB | + | + |
| 25 | ESC | + | + |
| 26 | SAL | + | + |
| 27 | CEL | + | + |
| 28 | MAL | − | + |
| 29 | LAC | + | + |
| 30 | MEL | − | − |
| 31 | SAC | − | − |
| 32 | TRE | + | + |
| 33 | INU | − | − |
| 34 | MLZ | + | + |
| 35 | RAF | − | − |
| 36 | AMD | − | − |
| 37 | GLYG | − | − |
| 38 | XLT | − | − |
| 39 | GEN | − | + |
| 40 | TUR | − | − |
| 41 | LYX | − | − |
| 42 | TAG | + | + |
| 43 | DFUC | − | − |
| 44 | LFUC | − | + |
| 45 | DARL | − | − |
| 46 | LARL | − | − |
| 47 | GNT | − | − |
| 48 | 2KG | − | − |
| 49 | 5KG | + | + |

“+”: positive reaction;
“−”: negative reaction.

Where, No. 0 tube is a blank control.

(5) Morphology of Gram-Stained Mycelium of CMU-pb-7

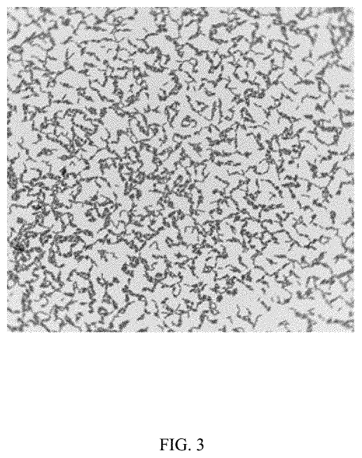

The single colony of CMU-pb-7 on the MRS agar plate is selected and smeared on a glass slide containing 10 μL of sterile normal saline; after the glass slide is dried, the external flame of an alcohol lamp is fixed; after the glass slide is cooled, Gram staining is performed, and the morphology of the mycelium is observed under an oil lens; and the results are shown in FIG. 3, which show that CMU-pb-7 is a purple rod-shaped Gram-positive bacterium arranged singly, in pairs or in short chains.

### Embodiment 2 Establishment of High-Fat Diet Mouse Model

24 SPF grade male C57BL/6 mice aged 6 to 8 weeks and weighing 19 to 21 g are selected. The mice are randomly divided into 3 groups with 8 mice in each group, which are respectively a control group (normal diet, ND): treated by gavage with a normal diet (manufacturer: Beijing Keao Xieli Feed Co., Ltd., name: Growth and Reproduction Feed for Rats and Mice)+0.2 mL of normal saline; a model group (high-fat diet, HFD): treated by gavage with a high-fat diet (manufacturer: Jiangsu Xietong Pharmaceutical Bio-engineering Co., Ltd., name: 60% Kcal High Fat Diet, article No.: XTHF60)+0.2 mL of normal saline; and a HFD+CMU-pb-7 group: treated by gavage with a high-fat diet+0.2 mL of 1×10⁹ CFU/mL CMU-pb-7. Each group of mice are treated by gavage at a fixed time every day. The mice are treated for 9 continuous weeks, and executed by cervical dislocation after eyeball blood collection. Samples of the liver and the ileum of the mice are reserved for later detection and analysis.

### Embodiment 3 Assessment of Antioxidant Indexes of High-Fat Diet Mice

(1) Liver Antioxidant Factor Detection of High-Fat Diet Mice

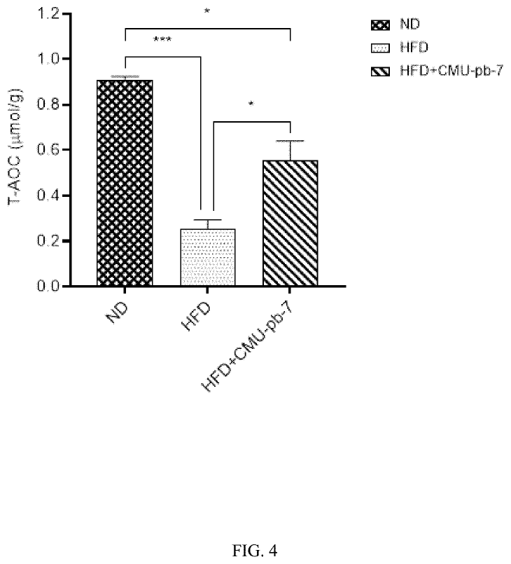

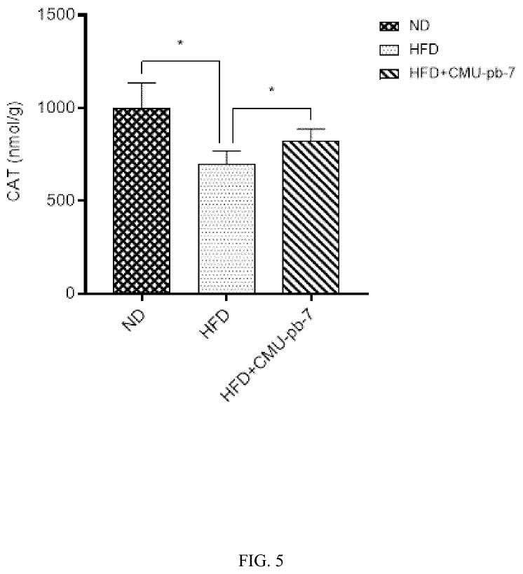

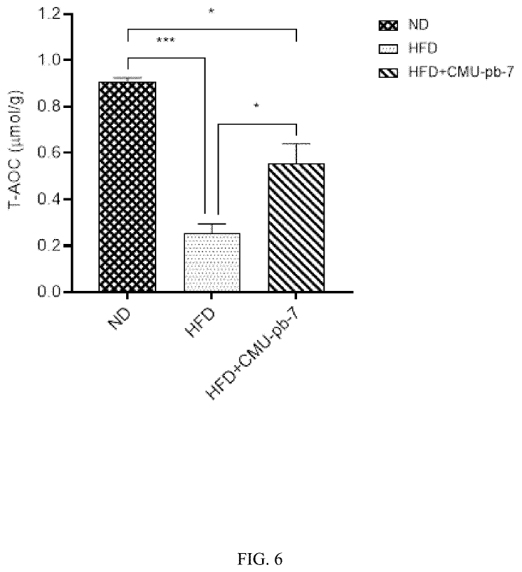

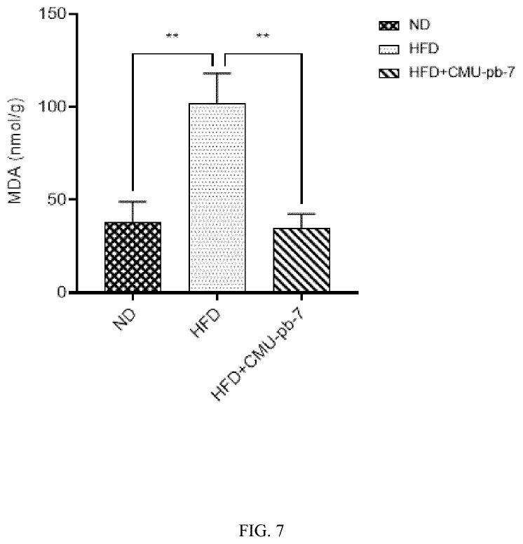

0.1 g of mouse liver tissue is added into 1 mL of precooled lysate (Solarbio), homogenized at 4° C. with a speed of 10000 r/min, and centrifuged for 5 min; and the supernatant is collected. Levels of antioxidant factors in the liver tissue are respectively determined by a reductive glutathione (GSH) assay kit, a hydrogen peroxide (CAT) activity assay kit and a total antioxidant capacity (T-AOC) assay kit, and levels of lipid peroxidation in the liver tissue are determined by a malondialdehyde (MDA) assay kit. The results are shown in FIG. 4 to FIG. 7, which show that: compared with the ND group, the T-AOC, GSH and CAT levels in liver of the HFD group are significantly decreased, while the MDA level is increased (P<0.05), indicating that the antioxidant capacity of the liver tissue is decreased; and compared with the HFD group, the GSH, CAT and T-AOC levels in the HFD+CMU-pb-7 group are significantly increased, while the MDA level is decreased (P<0.05), indicating that CMU-pb-7 can improve the antioxidant capacity, reduce the oxidative stress level and protect the liver tissue.

(2) Liver Morphological Observation of High-Fat Diet Mice

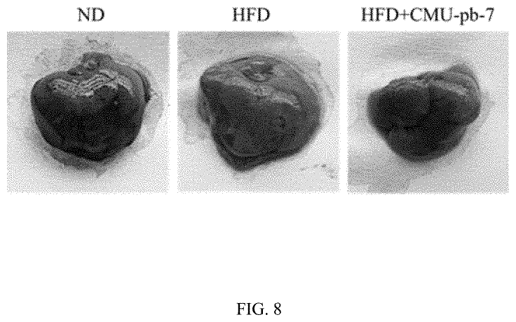

After each mouse is dissected, the liver is rinsed with PBS, and the volume, hardness, smoothness and fatty change of the liver of the mice are observed by naked eyes and recorded by taking photographs. The results are shown in FIG. 8, which show that: compared with the ND group, the volume of the liver of the mice in the HFD group is larger, the texture is hard, the cut surface is greasy, and fatty change is observed; and compared with the HFD group, the volume of the liver of the mice in the HFD+CMU-pb-7 group is smaller, the texture is soft, the cut surface is smooth, and no fatty change is observed, indicating that CMU-pb-7 can improve the antioxidant capacity and relieve fatty change.

(3) Ileum Morphological Observation of High-Fat Diet Mice

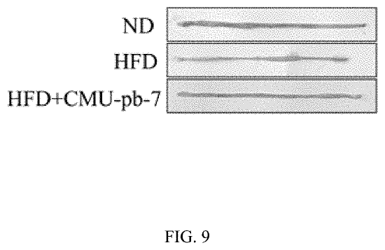

After each mouse is dissected, the ileum is rinsed with PBS, and the length, tissue adhesion, and swelling of the mouse ileum are observed by naked eyes and recorded by taking photographs. The results are shown in FIG. 9, which show that: compared with the ND group, the length of the ileum of the mice in the HFD group is shortened, and tissue adhesion and swelling are observed; and compared with the HFD group, the length of the ileum of the mice in the HFD+CMU-pb-7 group is longer, and no tissue adhesion or swelling is observed, indicating that CMU-pb-7 can relieve ileum injury.

(4) Ileum Tissue HE Staining of High-Fat Diet Mice

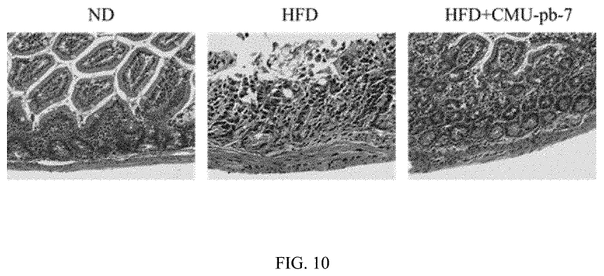

\textcircled{1} The ileum tissue of the mice in each group is fixed with 4% formaldehyde solution, embedded in paraffin and sliced. \textcircled{2} The paraffin slices are put into an oven at 60° C. for 1 h and soaked in xylene for 5 min; soaked in anhydrous ethanol, 95% ethanol, 85% ethanol and 75% ethanol for 3 min respectively; and then rinsed with running water and spin-dried. \textcircled{3} After stained by hematoxylin for 5 min, the paraffin slices are rinsed with running water, differentiated by 1% hydrochloric acid alcohol, and rinsed again with running water; stained by eosin for 1 min; and then soaked in 75% ethanol, 85% ethanol, 95% ethanol and anhydrous ethanol for 1 min respectively, put into an oven at 60° C. for 5 min, and mounted with neutral resin. \textcircled{4} Observation is made under a microscope and photographs are taken. The histopathological changes of the ileum tissue of the mice in each group are observed and compared under an optical microscope, and the results are shown in FIG. 10, which show that: the structure of the ileum tissue of the mice in the ND group is complete; the intestinal wall structure of the ileum tissue of the mice in the HFD group is incomplete, intestinal villi are broken and absent, epithelial cells are shed, inflammatory cell infiltration is observed, and the injury of the ileum tissue is serious; the intestinal wall structure of the ileum tissue of the mice in the HFD+CMU-pb-7 group is complete, no intestinal villus is broken or absent, epithelial cells are complete and not shed, inflammatory cell infiltration is reduced, and the injury of the ileum tissue is relieved, indicating that CMU-pb-7 can regulate the oxidative stress level in the intestinal tract and relieve the oxidative damage of the ileum tissue.

(5) Liver Index Detection of High-Fat Diet Mice

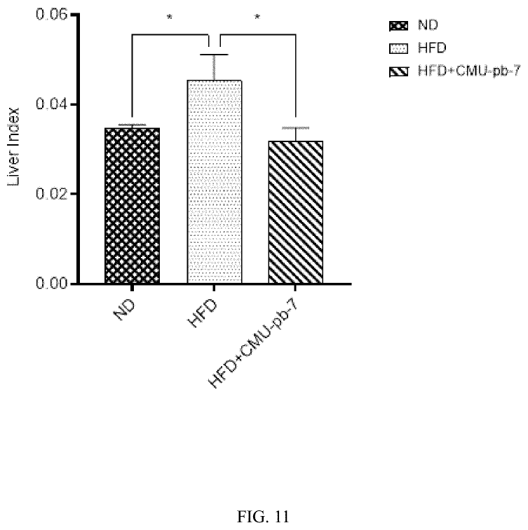

The mass of each mouse before dissection is denoted as M2, and the mass of the liver tissue of each mouse after dissection is denoted as M1. After weighing, the liver index of each mouse is calculated, and the liver index=M1/M2. The results are shown in FIG. 11, which show that: compared with the ND group, the liver index of the mice in the HFD group is increased (P<0.05); and compared with the HFD group, the liver index of the mice in the HFD+CMU-pb-7 group is decreased (P<0.05), indicating that CMU-pb-7 can relieve the changes of the volume and weight of the liver of the mice.

(6) Ileum Histological Injury Scores of High-Fat Diet Mice

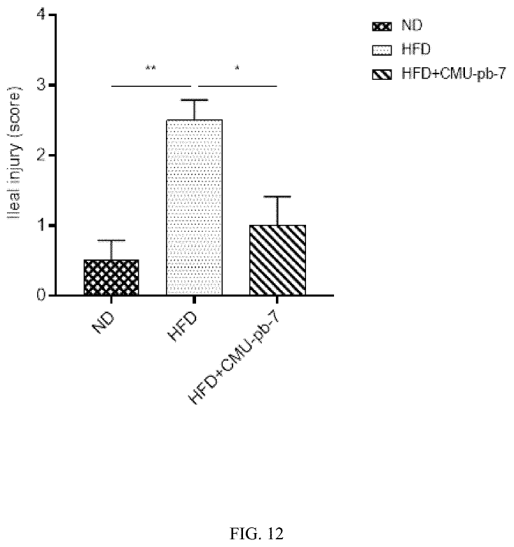

Ileum histological injury scores: 0, normal intestinal mucosa; 1, mild edema, telangiectasis and hyperemia under the epithelium at the top end of the intestinal mucosa; 2, the gap between the epithelial cells and the lamina propria of the intestinal mucosa is increased; 3, the top end of part of the lamina propria of the intestinal mucosa is exposed, and the epithelial cells are shed; 4, the lamina propria of the mucosa is exposed or the structure of glandular epithelium is absent, and telangiectasis and hyperemia occur, which may also be accompanied by inflammatory cell infiltration of the lamina propria; and 5, intestinal mucosa bleeding, ulcer and lamina propria disintegration. The assessment results are shown in FIG. 12, which show that: the ileum tissue injury scores of the mice in the HFD+CMU-pb-7 group are significantly lower than those in the HFD group, indicating that CMU-pb-7 can relieve the oxidative damage of the ileum tissue of high-fat diet mice.

The above description of the disclosed embodiments enables those skilled in the art to realize or use the present invention. Many modifications to these embodiments will be apparent to those skilled in the art. The general principle defined herein can be realized in other embodiments without departing from the spirit or scope of the present invention. Therefore, the present invention will not be limited to these embodiments shown herein, but will conform to the widest scope consistent with the principle and novel features disclosed herein.

What is claimed is:

1. A medical cell CMU-pb-7, having a collection number of CCTCC NO: M 2022220.

2. An application of the medical cell CMU-pb-7 according to claim 1 in preparation of antioxidant drugs.
